(19) United States
(12) Patent Application Publication
(10) Pub. No.: US 2006/0127931 A1
(43) Pub. Date: Jun. 15, 2006

(54) Particle detector with waveguide light confinement

(76) Inventors: Bradley Schmidt, Ithaca, NY (US); Michal Lipson, Ithaca, NY (US); Vilson Rosa de Almeida, Sao Jose dos Campos (BR)

Correspondence Address: SCHWEGMAN, LUNDBERG, WOESSNER & KLUTH, 121 S. 8TH STREET, SUITE 1600, MINNEAPOLIS, MN, 55402, US

(21) Appl. No.: 11/274,938

(22) Filed: Nov. 15, 2005

Related U.S. Application Data
(60) Provisional application No. 60/627,938, filed on Nov. 15, 2004.

(51) Int. Cl. C12Q 1/68; C12M 1/34; G01N 33/551

(52) U.S. Cl. 435/6; 435/287.2; 977/924; 436/524

(57) ABSTRACT

A strong light confining nano-cavity in a photonic structure enhances the effective extinction cross-section of metal nano-particles. As a result of strong light confinement, precisely where the particle is located, the presence of a single metal nano-particle with a diameter as small, or smaller than 10 nm may be detected by measuring the decrease in transmission of light propagating through the photonic structure. In one embodiment, gold particles may be used as a sensing probe due to their large extinction coefficient in a wavelength range of (1450-1600 nm) and their mature use as labels in biosensing systems. The nanoparticles may be specifically bound to various analytes such as DNA, RNA, proteins and antigens.

### RELATED APPLICATION

This application claims priority to U.S. Provisional Application Ser. No. 60/627,938 (entitled NANO-CAVITY IN A SILICON WAVEGUIDE FOR ULTRA SENSITIVE NANO-PARTICLE DETECTION, filed Nov. 15, 2004) which is incorporated herein by reference.

### GOVERNMENT FUNDING

The invention described herein was made with U.S. Government support under cooperative agreements DMR-0079992, DMR-0120967 with the National Science Foundation and Grant Number ECS-9731293 awarded by the National Science Foundation. The United States Government has certain rights in the invention.

### BACKGROUND

There is a growing need for the development of environmental, health safety, and clinical microfabricated biosensors for many analytes such as DNA, RNA, proteins, antigens, and other bio-molecules, which allow for lower cost, smaller sample volumes, massive parallelism and ultra high sensitivity. Many of these biosensing systems rely on binding the analyte to individual label particles, such as quantum dots, gold particles, and fluorescent dye molecules. Current systems for ultra sensitive bio-detection using these labels are either complex, large, or lack the desired level of sensitivity. The challenge of improving the sensitivity of integrated systems is due to the low cross-section (emission or absorption) of the labels that are often bound to the analyte.

### SUMMARY

A strong light confining nano-cavity in a photonic structure enhances the effective extinction cross-section of metal nano-particles. As a result of strong light confinement, precisely where the particle is located, the presence of a single metal nano-particle with a diameter as small or smaller than 10 nm may be detected by measuring the decrease in transmission of light propagating through the photonic structure. In one embodiment, gold particles may be used as a sensing probe due to their large extinction coefficient in a wavelength range of (1450-1600 nm) and their mature use as labels in biosensing systems.

In one embodiment, the photonic structure comprises a one-dimensional photonic crystal consisting of a high index contrast silicon waveguide having nanometer size dimensions with nm range diameter holes filled with a lower index material in the waveguide to create Distributed Bragg Reflectors (DBR's) on either side of a cavity. A small nm range diameter nano-cavity filled hole having a low index is embedded in the center of the cavity. The addition of this defect at the center of the cavity creates a local discontinuity in the field, increasing the strength of the field in the center of the cavity.

### BRIEF DESCRIPTION OF THE DRAWINGS

FIG. 1 is a block perspective diagram of a light confining waveguide having a field enhancing structure according to an example embodiment.

FIG. 2 is a cross section diagram illustrating a light confining waveguide having a field enhancing structure and fluidic channel according to an example embodiment.

FIGS. 3A and 3B illustrate finite difference time domain simulations of the waveguide of FIG. 1.

FIG. 4 illustrates a finite difference time domain simulation of varying field enhancing structure dimensions according to an example embodiment.

FIG. 5 illustrates normalized transmission as a function of a number of particles in a sensing area according to an example embodiment.

### DETAILED DESCRIPTION

In the following description, reference is made to the accompanying drawings that form a part hereof, and in which is shown by way of illustration specific embodiments which may be practiced. These embodiments are described in sufficient detail to enable those skilled in the art to practice the invention, and it is to be understood that other embodiments may be utilized and that structural, logical and electrical changes may be made without departing from the scope of the present invention. The following description is, therefore, not to be taken in a limited sense, and the scope of the present invention is defined by the appended claims.

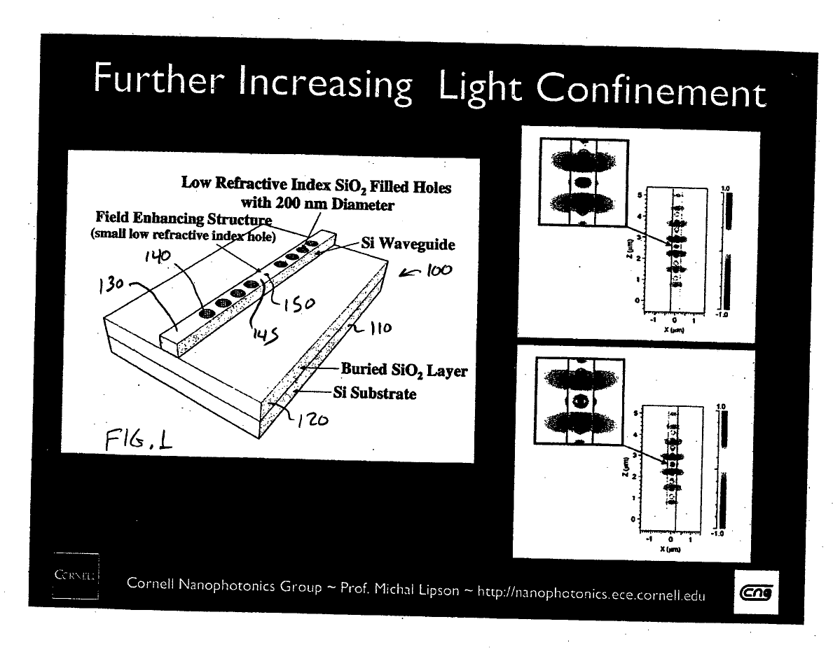

In FIG. 1, an example embodiment, of a photonic structure 100 supported by substrate 110 and insulating layer 120, such as a buried SiO₂ layer. Photonic structure 100 comprises a one-dimensional photonic crystal consisting of a high index contrast silicon waveguide 130 (approximately 450 nm wide, 250 nm in height) with approximately 200 nm diameter holes 140 filled with a lower index material (silicon dioxide with n=1.46) in the waveguide to create Distributed Bragg Reflectors (DBR's) on either side of a 910 nm long cavity 145. The size and number of holes and distance between the holes may be varied, such as by a function of wavelength, to provide desired reflective characteristics. A small 100 nm diameter SiO₂ nano-cavity 150 (filled hole) is embedded approximately in the center of the cavity 145. The addition of this defect at the center of the cavity creates a local discontinuity in the field, increasing the strength of the field in the center of the cavity. It should be noted that the small hole 150 need not be filled with SiO2 to provide such a defect. Unfilled holes, or holes filled with a higher index material may also provide such a defect to create a local discontinuity in the field.

The photonic structure 100 may be fabricated in a number of different ways. One example manner of fabricating photonic structure 100 comprises using Silicon on Insulator (SOI) wafers with 250 nm of crystalline silicon 130 on top of a three microns thick buried oxide layer 120. The structure may be defined using electron-beam lithography using FOx-12 spin-on glass as a negative resist and etch mask and etched by Chlorine based reactive ion etching (RIE). The holes 140 may be filled and the structure clad with SiO₂ using plasma enhanced chemical vapor deposition.

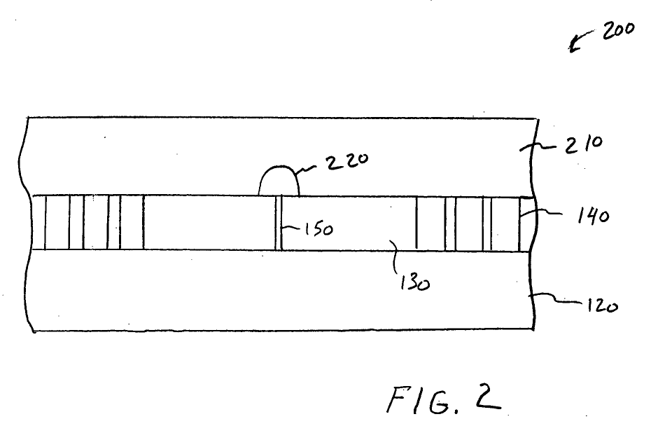

FIG. 2 illustrates a photonic structure 200 clad with SiO₂ as shown at 210. Parts of photonic structure 200 that are similar to photonic structure 100 are similarly numbered. In one embodiment, the cladding 210 comprises a fluidic channel 220 formed therein. Channel 220 is shown in FIG. 2 as substantially transversely intersecting the local discontinuity 150, and may be used to deliver metal particles, which affects light transmission about the discontinuity. The light confining properties of the local discontinuity enhance the effective extinction cross section of the metal nano-particles as they cross proximate the discontinuity by way of the fluidic channel 220. The area of the channel proximate the discontinuity may be referred to as a sensing area.

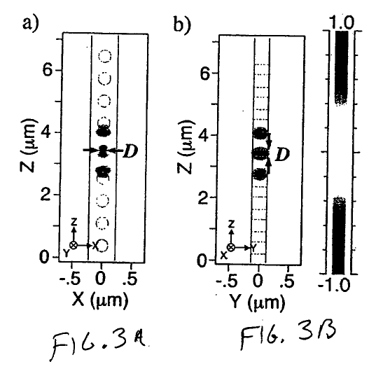

In FIGS. 3A and 3B, 3-D Finite Difference Time Domain (FDTD) simulations of the structure are illustrated. The illustrations show a TE-like mode field profile of the field inside the micro-cavity with 100 nm diameter nano-cavity D (n=1.46) at resonance wavelength λ=1.568 microns. FIG. 3A is top view center cross-section, and FIG. 3B is a side view center cross-section. Due to the light confinement in the cavity and the presence of the small nano-cavity, a strong field (TE-like mode) is present in the center of the device, which is approximately 315 times stronger than the field inside the core of the regular waveguide. The field enhancement induces a strong enhancement of the effective extinction cross-section of the metal nano-particle.

A side cross-section of the 3-D FDTD simulations (FIG. 3B) shows that the field at the top surface of the central nano-cavity is relatively strong, approximately half the magnitude of the field at the center depth of the nano-cavity. This indicates that the top surface of the cavity can be used as a sensor without the need to embed the nano-particles inside the waveguide in order to be detected. The small out of plane radiation also enables performance of relatively accurate 2-D simulations on the structure as opposed to 3-D. In one embodiment, the device may be chosen to have a quality factor Q=190. This Q enhances the cross-section of the chosen size of metal nano-particle in order to allow the clear detection of single particles, without being too sensitive to fluctuations in environmental conditions, and to the exact position of the particle.

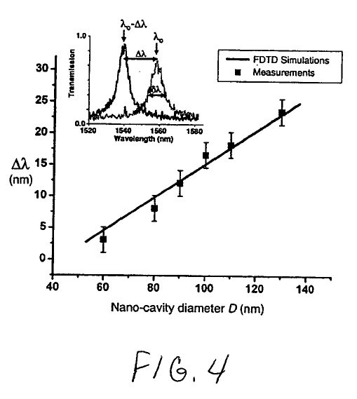

In order to verify that the field in the device is indeed localized in the center of the cavity and to investigate the degree of confinement, the spectral response of several fabricated devices may be measured. In one embodiment, all devices have the same dimensions except for varying the diameter D of the central nano-cavity. FIG. 4 is a graph illustrating the relative shift of the spectral resonances of the micro-cavities due to the change in the diameter of the nano-cavity. The inset of FIG. 4 shows transmission spectra of two devices, each with a different nano-cavity diameter. The quality factor Q of the devices (λₒ/δλ, equal to the ratio of the energy stored in the device at resonance to the energy lost per cycle of oscillation is equal to approximately 182.3. The strong dependence of the spectra due to the very small variation in size of the nano-cavity indicates that the field is strongly confined within this small region. Experimental results appear to closely match 2-D simulation results, showing that the resonance shift due to the varying nano-cavity diameters correspond to those predicted by FDTD calculations (solid line) of Δλ/D=0.263.

Transmission losses due to the presence of gold metal nano-particles may be measured using the same devices with a 100 nm diameter nano-cavity. Various size particles and particles of different strongly absorbing material such as other metals like silver, etc. The signal would be stronger or weaker depending on material and size.

Due to the use of a top cladding for optimal operation of fiber to waveguide couplers the upper cladding was only removed above the cavity devices. The upper cladding was removed by patterning 20 micron diameter holes using photolithography. It was etched close to the surface of the cavity using CHF₃ based RIE, followed by highly selective HF wet chemistry in order to remove the remaining cladding down to the top surface above the cavity without allowing possible roughness from the RIE process. Once the cavities were exposed, the transmission through the devices was measured with water and various depositions of colloidal gold particles on top of the cavities. These depositions were achieved by placing small amounts of water-based solutions of 10 nm gold particles (1.9×10¹³ particles per ml) on top of the devices and allowing them to dry by evaporation. Each deposition step deposited approximately 30.0 particles per micron² on the entire structure, corresponding to approximately 1.25±0.2 particles in the sensing area of 0.04 μm² per additional deposition step. After each evaporation, the device was again covered in water. Careful placement allowed the particles to remain settled on the surface while transmission measurements were made. The optical sensing area may be calculated as the area of the top surface of the device weighted by the field intensities in each region.

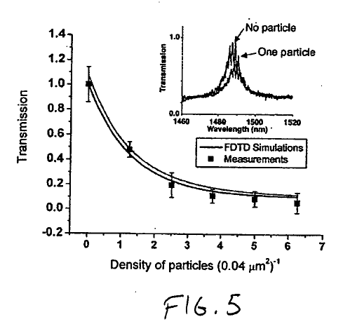

FIG. 5 shows the measured transmission as a function of the number of nano-particles deposited in the optical sensing area of 0.04 μm². The error bars represent deviation from the average measured value due to the processing and temperature variation in our experiments. The inset of FIG. 5 shows the spectra measured for no particles on the structure and for one particle in the optical sensing area. A strong decrease in transmission may be observed with increasing number of particles, with a drop in transmission of as much as 52% for the addition of the first deposition of particles on the sensing area.

In order to theoretically analyze the structure as a sensor, the presence of gold nano-particles in the 2-D FDTD simulations assume that they are bound to the top surface of the cavity. These simulations assumed that the top oxide cladding had been replaced with water. The top of the structure may be assumed to be unclad and covered by water (n=1.33, k=1.48×10⁻⁴, at 25° C. and at a wavelength of 1550 nm). FIG. 5 shows that a large decrease in expected transmission intensity per additional particle may be observed (solid black line) in agreement with experimental results. The error bars in the theoretical curve represent the deviation from the average value due to variations in the particle position. FDTD simulations also predict low losses due to scattering from the particle on the top surface of the waveguide. A difference in forward scattering and lateral scattering between the device with and without the particle is less than 0.02%, and 0.6% respectively, confirming that the transmission decrease is mainly due to the absorption by the particle.

Due to the characteristic modal volume of this cavity along with the presence of the low index nano-cavity in the center of the micro-cavity, the gradient of the field is small over the area of the selected sensing region. Therefore similar results were achieved when the particles in the simulation were randomly placed away from the center, but still on the top surface of the nano-cavity, resulting in less than a 2% change in absorption losses as compared to when the particles were placed only in the very center of the sensing area. This change is shown as the grey solid line in FIG. 5.

### CONCLUSION

A micron-size planar silicon photonic device may be used to detect ultra low concentrations of metal nano-particles. A high detection sensitivity is achieved by using a strong light confining structure that enhances the effective extinction cross-section of metal nano-particles. 10 nm diameter gold particles with a density of fewer than 1.25 particles per 0.04 μm² may be detected. Such a device may detect the presence of single metal nano-particles specifically bound to various analytes, enabling ultra-sensitive detection of analytes including DNA, RNA, proteins, and antigens.

The Abstract is provided to comply with 37 C.F.R. §1.72(b) to allow the reader to quickly ascertain the nature and gist of the technical disclosure. The Abstract is submitted with the understanding that it will not be used to interpret or limit the scope or meaning of the claims.

1. A device comprising:
a high index contrast waveguide;
a pair of distributed Bragg reflectors disposed along the waveguide, separated by a desired length of the waveguide; and
a light confining discontinuity in the waveguide positioned in the desired length of the waveguide between the reflectors.

2. The device of claim 1 wherein the discontinuity comprises a nano-cavity hole.

3. The device of claim 2 herein the nano-cavity hole is filled with SiO₂.

4. The device of claim 3 wherein the nano-cavity hole is approximately 100 nm in diameter.

5. The device of claim 1 wherein the reflectors are formed of approximately 200 nm diameter holes filled with lower index of refraction material than the waveguide.

6. The device of claim 5 wherein the holes are filled with SiO₂.

7. The device of claim 1 and further comprising a fluidic channel proximate the discontinuity and at least partially orthogonal to the waveguide.

8. The device of claim 1 and further comprising low refractive index cladding covering the waveguide.

9. The device of claim 8 and further comprising a fluidic channel formed in the cladding and crossing the waveguide proximate the discontinuity.

10. The device of claim 9 wherein metal nanoparticles on a top surface of the discontinuity affect light passing through the waveguide.

11. A method comprising:
providing metal nanoparticles on a surface of a nano-discontinuity of a nanometer size high index contrast waveguide;
reflecting light with distributed Bragg reflectors on opposite sides of the nano-discontinuity; and
measuring differences in light transmitted through the waveguide representative of the metal nanoparticles.

12. The method of claim 11 wherein the nanoparticles are specifically bound to various analytes.

13. The method of claim 12 wherein the analytes are selected from the group consisting of DNA, RNA, proteins and antigens.

14. The method of claim 12 wherein the nanoparticles comprise gold.

15. The method of claim 14 wherein the gold nanoparticles are approximately 10 nm in diameter.

16. The method of claim 11 wherein the metal nanoparticles are provided on the surface of the nano-discontinuity by evaporation.

17. The method of claim 11 wherein the discontinuity is a low refractive index SiO₂ filled hole in the waveguide.

18. The method of claim 17 wherein the hole is approximately 100 nm in diameter.

19. A device comprising:
a nanometer size high index contrast silicon waveguide;
means for creating reflectors; and
means for creating light confinement between the reflectors.

20. The device of claim 19 and further comprising means for providing metal nanoparticles proximate the light confinement to modulate light passing through the waveguide.

21. A particle detector comprising:
means for enhancing light confinement in a desired section of nanometer size high index waveguide;
means for directing the flow of an analyte of interest proximate the desired section; and
means for measuring light passing through the waveguide in the desired section.

22. A device comprising:
a waveguide; and
a light confining discontinuity in the waveguide.

23. The device of claim 22 and further comprising a fluid channel positioned adjacent the light confining discontinuity.

24. The device of claim 22 and further comprising a light source coupled to inject light into the waveguide and a light detector that detects light after it has passed through the light confining discontinuity.
